(19) United States
(12) Patent Application Publication
(10) Pub. No.: US 2011/0137098 A1
(43) Pub. Date: Jun. 9, 2011

(54) PROCESS FOR ISOMERIZING A SATURATED HYDROCARBON

(75) Inventors: STEFFEN TSCHIRSCHWITZ, Mannheim (DE); Stephan Deuerlein, Ludwigshafen (DE); Jochen Bürkle, Mannheim (DE); Markus Schmitt, Heidelberg (DE); Steffen Oehlenschläger, Antwerpen (BE); Kathrin Wissel-Stoll, Ludwigshafen (DE); Veronika Wloka, Mannheim (DE)

(73) Assignee: BASF SE, Ludwigshafen (DE)

(21) Appl. No.: 12/961,308

(22) Filed: Dec. 6, 2010

(30) Foreign Application Priority Data
Dec. 7, 2009 (EP) 09178210.2

(51) Int. Cl. C07C 5/29

(52) U.S. Cl. 585/374; 585/747

(57) ABSTRACT

A process for isomerizing a saturated hydrocarbon, by performing the isomerization in the presence of a superacidic ionic liquid comprising an organic cation and an inorganic anion, where the anion is a superacidic aluminum trichloride-Lewis base adduct, and of an olefin.

The invention relates to a process for isomerizing a saturated hydrocarbon.

The isomerization of saturated hydrocarbons (paraffins) to the corresponding branched isomers is an important process, for example, for increasing the research octane number (RON) of gasoline, in order to improve the combustion properties thereof.

Branched cyclic hydrocarbons can isomerize with ring enlargement to less branched cyclic hydrocarbons; one example is the rearrangement of methylcyclopentane (MCP) to cyclohexane. These reactions are catalyzed by strong Lewis acids or strong Brønsted acids.

The isomerization of saturated hydrocarbons with solid aluminum chloride has been known for some time. A frequently used accelerating additive is HCl (e.g. U.S. Pat. No. 2,493,567, U.S. Pat. No. 3,233,001, U.S. Pat. No. 5,202,519). Problems in these processes are the long-term stability of solid aluminum chloride and the removal thereof.

US 2003/0109767 A1 (Vasina et al.) reports that ionic liquids consisting of a nitrogen-containing heterocyclic or aliphatic cation and an anion which derives from a metal halide can be utilized for isomerization of paraffins in the direction of more highly branched paraffins at relatively low temperatures.

Cyclic hydrocarbons with a tertiary carbon atom as additives, such as methylcyclohexane and dimethylcyclopentane, according to EP 1 403 236 A1 (Haldor Topsoe A/S), increase the selectivity with regard to the formation of more highly branched hydrocarbons from less branched or unbranched hydrocarbons.

Ionic liquids consisting of n-butylpyridinium chloride and aluminum chloride can be utilized in order to isomerize methylcyclopentane and cyclohexane: V. A. Ksenofontov, T. V. Vasina, Y. E. Zubarev, L. M. Kustov, React. Kinet. Catal. Lett. 2003, Vol. 80 (2), pages 329-335.

It was an object of the present invention to provide, overcoming the disadvantages of the prior art, an improved, economically viable process for isomerizing a saturated hydrocarbon.

The preparation process should additionally be particularly simple and economically viable, and should provide the process product (a saturated hydrocarbon with the same empirical formula) in high yields, more particularly in high space-time yields (STY). Accordingly, a process has been found for isomerizing a saturated hydrocarbon, which comprises performing the isomerization in the presence of a superacidic ionic liquid comprising an organic cation and an inorganic anion, where the anion is a superacidic aluminum trichloride-Lewis base adduct, and of an olefin.

It has been recognized in accordance with the invention that the isomerization of a saturated hydrocarbon can be greatly accelerated when the isomerization is performed in the presence of a superacidic ionic liquid comprising aluminum chloride and in the presence of an olefin as a catalyst.

The process according to the invention is therefore superior to conventional processes because the reaction equilibria are attained significantly more rapidly.

The olefin is preferably a linear or branched and/or cyclic C₂₋₁₄-olefin, especially a linear or branched and/or cyclic C₂₋₁₀-olefin, very particularly a linear or branched and/or cyclic C₂₋₇-olefin.

Preferred olefins are ethene, propene, 1-butene, cis-butene-2, trans-butene-2, isobutene 3-methyl-1-butene, 1-pentene, 2-methyl-1-butene, trans-pentene-2, cis-pentene-2,2-methyl-2-butene, cyclopentene, 4-methyl-1-pentene, 3-methyl-1-pentene, methylpentadiene, 2-methyl-1-pentene, trans-hexene-2, cis-hexene-2,2-methyl-2-pentene, 3-methylcyclopentene, 3-methyl-cis-pentene-2, cis-hexene-2,3-methyl-trans-pentene-2, 4,4-dimethyl-trans-pentene-2,1-methylcyclopentene, cyclohexene and trans-heptene-3, especially 3-methyl-1-butane, 1-pentene, 2-methyl-1-butene, trans-pentene-2, cis-pentene-2,2-methyl-2-butene, cyclopentene, 2-methyl-1-pentene, 2-methyl-2-pentene, 3-methylcyclopentene, cis-hexene-2,3-methyl-trans-pentene-2,1-methylcyclopentene, cyclohexene and trans-heptene-3.

The olefin is preferably a monoolefin.

The olefin is most preferably ethene, 2-methyl-1-butane, 2-methyl-2-butene or 1-methyl-cyclopentene.

The isomerization is preferably performed in the presence of 0.01 to 5% by weight, particularly 0.1 to 3% by weight, especially >0.1 to 2% by weight, more particularly 0.5 to 1.5% by weight, of the olefin, based in each case on the saturated hydrocarbon used.

The hydrocarbon to be isomerized is preferably a linear or branched and/or cyclic C₄₋₁₈ hydrocarbon, particularly a linear or branched and/or cyclic C₅₋₁₀ hydrocarbon, very particularly a linear or branched and/or cyclic C₅₋₈ hydrocarbon.

Examples of a linear hydrocarbon to be isomerized and of possible isomerization products (in brackets) are n-pentane (2-methylbutane, 1,1-dimethylpropane), n-hexane (2-methylpentane, 3-methylpentane, 2,2-dimethylbutane, 2,3-dimethylbutane, 3-ethylpentane), n-heptane (2-methylhexane, 3-methylhexane, 2,3-dimethylpentane), n-octane (isooctanes).

Examples of a branched (noncyclic) hydrocarbon to be isomerized and of possible isomerization products (in brackets) are 1-methylbutane (2-methylbutane), 1-methylpentane (2-methylpentane), 1-methylhexane (2-methylhexane).

Examples of a cyclic hydrocarbon to be isomerized and of possible isomerization products (in brackets) are cyclohexane (methylcyclopentane), cycloheptane (methylcyclohexane, MCH).

Examples of a branched and cyclic hydrocarbon to be isomerized and of possible isomerization products (in brackets) are methylcyclopentane (cyclohexane); 1,2-dimethylcyclopentane, 1,1-dimethylcyclopentane, 1,3-dimethylcyclopentane (methylcyclohexane).

The process according to the invention converts, for example, a secondary carbon atom in the hydrocarbon used to a tertiary carbon atom.

One example thereof is the isomerization of n-hexane to isohexane and of n-heptane to isoheptane. The isomerization here thus gives a more highly branched, saturated hydrocarbon as the product.

The inventive isomerization preferably converts a tertiary carbon atom of the hydrocarbon to a secondary carbon atom. The isomerization thus affords a less branched saturated hydrocarbon as the product.

One example thereof is the isomerization of methylcyclopentane (MCP) to cyclohexane (CH).

The hydrocarbon to be isomerized is preferably used in a concentration in the range from 1 to 90% by weight, particularly from 5 to 20% by weight, based in each case on the ionic liquid.

The isomerization is preferably performed at a temperature in the range from −20 to 150° C., particularly 40 to 100° C.

The isomerization is preferably performed at an absolute pressure in the range from 1 to 10 bar, particularly 1 to 6 bar.

Ionic liquids in the context of the present invention are preferably
(A) salts of the general formula (I)

[A]ₙ⁺[Y]ⁿ⁻ (I)
in which n is 1, 2, 3 or 4, [A]⁺ is a quaternary ammonium cation, an oxonium cation, a sulfonium cation or a phosphonium cation, and [Y]ⁿ⁻ is a mono-, di-, tri- or tetravalent anion;
(B) mixed salts of the general formulae (II)

[A¹]⁺[A²]⁺[Y]ⁿ⁻ (IIa) where n=2;

[A¹]⁺[A²]⁺[A³]⁺[Y]ⁿ⁻ (IIb) where n=3; or

[A¹]⁺[A²]⁺[A³]⁺[A⁴]⁺[Y]ⁿ⁻ (IIc) where n=4 and
where [A¹]⁺, [A²]⁺, [A³]⁺ and [A⁴]⁺ are each independently selected from the groups specified for [A]⁺, and [Y]ⁿ⁻ is as defined under (A); or
(C) mixed salts of the general formulae (III)

[A¹]⁺[A²]⁺[A³]⁺[M¹]⁺[Y]ⁿ⁻ (IIIa) where n=4;

[A¹]⁺[A²]⁺[M¹]⁺[M²]⁺[Y]ⁿ⁻ (IIIb) where n=4;

[A¹]⁺[M¹]⁺[M²]⁺[M³]⁺[Y]ⁿ⁻ (IIIc) where n=4;

[A¹]⁺[A²]⁺[M¹]⁺[Y]ⁿ⁻ (IIId) where n=3;

[A¹]⁺[M¹]⁺[M²]⁺[Y]ⁿ⁻ (IIIe) where n=3;

[A¹]⁺[M¹]⁺[Y]ⁿ⁻ (IIIf) where n=2;

[A¹]⁺[A²]⁺[M⁴]²⁺[Y]ⁿ⁻ (IIIg) where n=4;

[A¹]⁺[M¹]⁺[M⁴]²⁺[Y]ⁿ⁻ (IIIh) where n=4;

[A¹]⁺[M⁵]³⁺[Y]ⁿ⁻ (IIIi) where n=4; or

[A¹]⁺[M⁴]²⁺[Y]ⁿ⁻ (IIIj) where n=3 and
where [A¹]⁺, [A²]⁺ and [A³]⁺ are each independently selected from the groups specified for [A]⁺, [Y]ⁿ⁻ is as defined under (A), and [M¹]⁺, [M²]⁺, [M³]⁺ are each monovalent metal cations, [M⁴]²⁺ are divalent metal cations and [M⁵]³⁺ are trivalent metal cations.

The ionic liquids preferably have a melting point of less than 180° C. Additionally preferably, the melting point is within a range from −50° C. to 150° C., more preferably in the range from −20° C. to 120° C. and even more preferably less than 100° C.

The inventive ionic liquids are organic compounds, which means that at least one cation or an anion of the ionic liquid comprises an organic radical.

Compounds which are suitable for forming the cation [A]⁺ of ionic liquids are known, for example, from DE 102 02 838 A1. For instance, such compounds may comprise oxygen atoms, phosphorus atoms, sulfur atoms or especially nitrogen atoms, for example at least one nitrogen atom, preferably 1-10 nitrogen atoms, more preferably 1-5, even more preferably 1-3 and especially 1-2 nitrogen atoms. It is optionally also possible for further heteroatoms such as oxygen, sulfur or phosphorus atoms to be present. The nitrogen atom is a suitable carrier of the positive charge in the cation of the ionic liquid, from which, in equilibrium, a proton or an alkyl radical can then be transferred to the anion in order to obtain an electrically uncharged molecule.

In the case that the nitrogen atom is the carrier of the positive charge in the cation of the ionic liquid, a cation can first be obtained in the course of synthesis of the ionic liquids by quaternization on the nitrogen atom, for instance of an amine or nitrogen heterocycle. The quaternization can be effected by protonating or alkylating the nitrogen atom. According to the alkylating reagent used, salts with different anions are obtained. In cases in which it is impossible to form the desired anion directly in the quaternization, this can be done in a further synthesis step. Proceeding, for example, from an ammonium halide, the halide can be reacted with a Lewis acid to form a complex anion from halide and Lewis acid. Alternatively, the exchange of a halide ion for the desired anion is possible. This can be done by adding a metal salt with precipitation of the metal halide formed, by means of an ion exchanger or by displacing the halide ion with a strong acid (to release the hydrohalic acid). Suitable processes are described, for example, in Angew. Chem. 2000, 112, p. 3926-3945, and the literature cited therein.

Suitable alkyl radicals with which the nitrogen atom in the amines or nitrogen heterocycles can be quaternized, for example, are C₁-C₁₈-alkyl, preferably C₁-C₁₀-alkyl, more preferably C₁-C₆-alkyl and most preferably methyl. The alkyl group may be unsubstituted or have one or more identical or different substituents.

Preference is given to those compounds which comprise at least one five- to six-membered heterocycle, especially a five-membered heterocycle, which has at least one nitrogen atom and optionally an oxygen or sulfur atom, particular preference being given to those compounds which comprise at least one five- or six-membered heterocycle which has one, two or three nitrogen atoms and a sulfur or an oxygen atom, very particular preference to those having two nitrogen atoms. Additionally preferred are aromatic heterocycles.

Particularly preferred compounds are those which have a molar mass less than 1000 g/mol, most preferably less than 500 g/mol.

Additionally preferred are those cations which are selected from the compounds of the formulae (IVa) to (IVw),

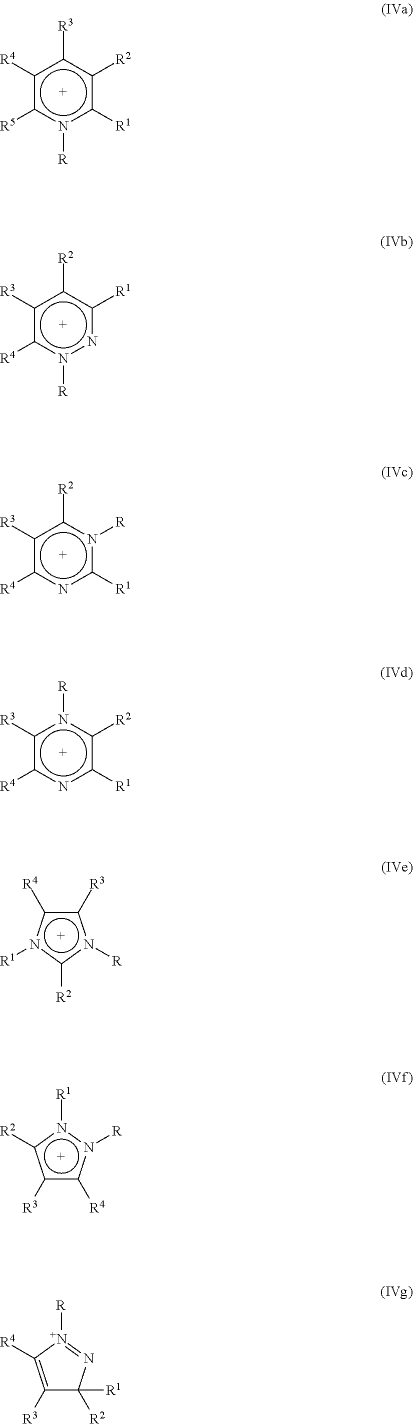

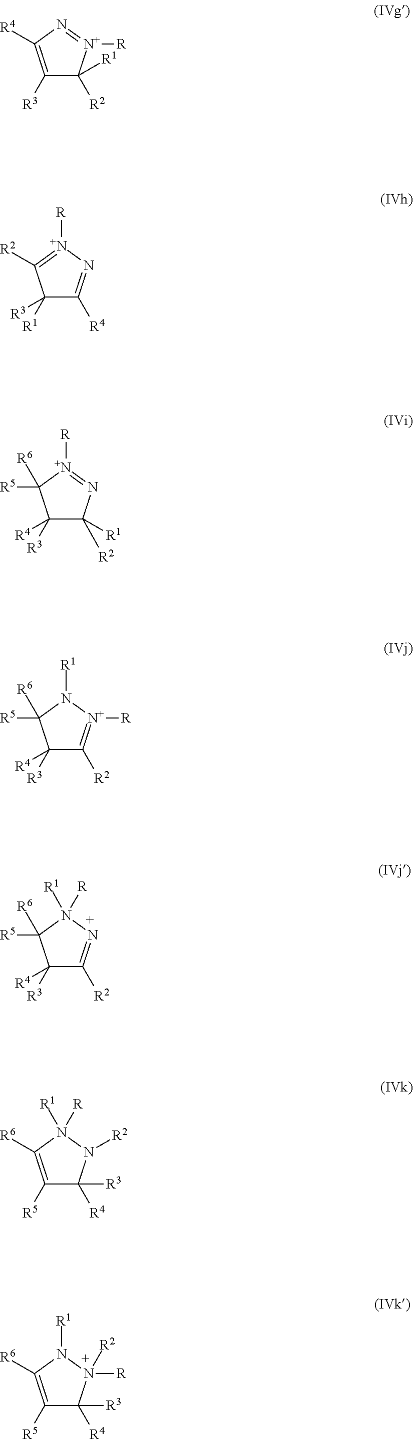

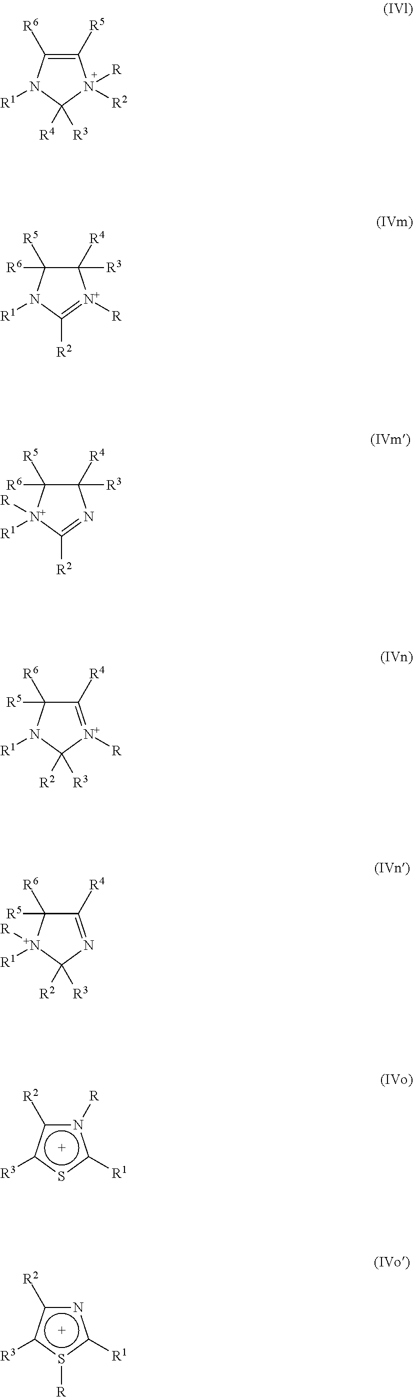

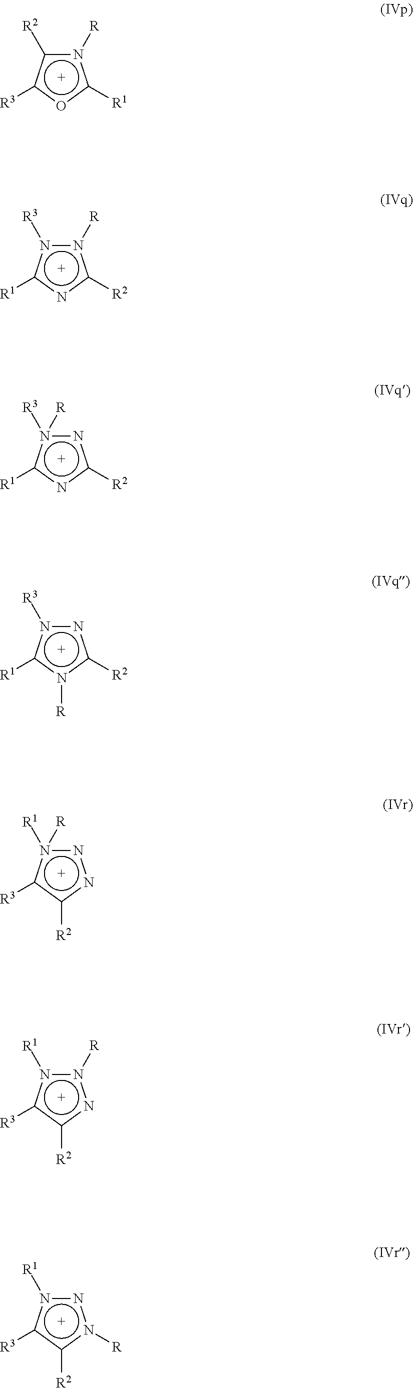

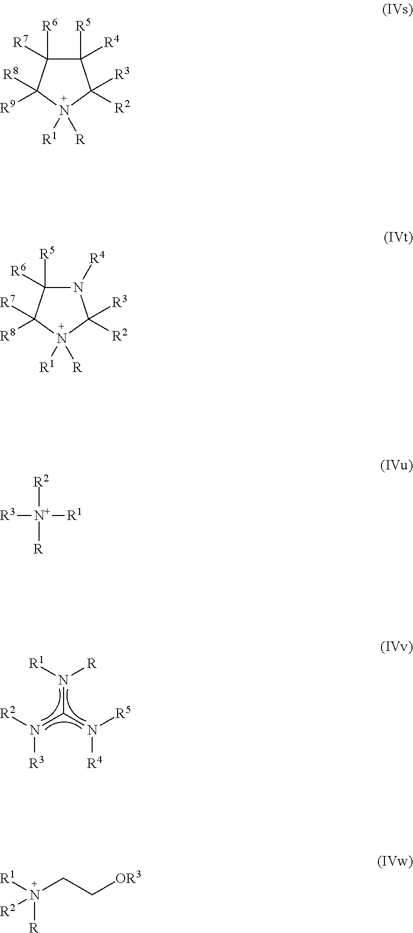

and oligomers which comprise these structures.

Further suitable cations are compounds of the general formulae (IVx) and (IVy)

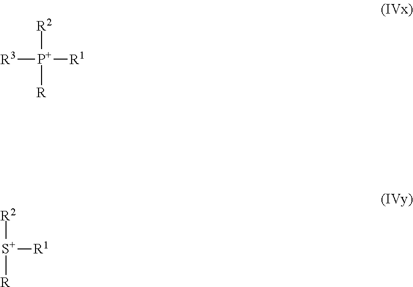

and oligomers which comprise this structure.

In the abovementioned formulae (IVa) to (IVy),
the R radical is hydrogen, a carbon-comprising organic, saturated or unsaturated, acyclic or cyclic, aliphatic, aromatic or araliphatic radical which is unsubstituted or interrupted by 1 to 5 heteroatoms or functional groups or substituted and has 1 to 20 carbon atoms; and
the R¹ to R⁹ radicals are each independently hydrogen, a sulfo group or a carbon-comprising organic, saturated or unsaturated, acyclic or cyclic, aliphatic, aromatic or araliphatic radical which is unsubstituted or interrupted or substituted by 1 to 5 heteroatoms or functional groups and has 1 to 20 carbon atoms, where the R¹ to R⁹ radicals which are bonded in the abovementioned formulae (IV) to a carbon atom (and not to a heteroatom) may additionally also be halogen or a functional group; or
two adjacent radicals from the R¹ to R⁹ series together are also a divalent, carbon-comprising organic, saturated or unsaturated, acyclic or cyclic, aliphatic, aromatic or araliphatic radical which is unsubstituted or interrupted or substituted by 1 to 5 heteroatoms or functional groups and has 1 to 30 carbon atoms.

Useful heteroatoms in the definition of the R and R¹ to R⁹ radicals are in principle all heteroatoms which are capable of formally replacing a —CH₂—, a —CH═, a —C≡ or a ═C═ group. When the carbon-comprising radical comprises heteroatoms, preference is given to oxygen, nitrogen, sulfur, phosphorus and silicon. Preferred groups include especially —O—, —S—, —SO—, —SO₂—, —NR′—, —N═, —PR′—, —PR′₂ and —SiR′₂—, where the R′ radicals are the remaining part of the carbon-comprising radical. The R¹ to R⁹ radicals may, in the cases in which they are bonded to a carbon atom (and not to a heteroatom) in the abovementioned formulae (IV), also be bonded directly via the heteroatom.

Useful functional groups are in principle all functional groups which may be bonded to a carbon atom or a heteroatom. Suitable examples include —OH (hydroxyl), ═O (especially as a carbonyl group), —NH₂ (amino), —NHR′, —NR₂′═NH (imino), —COOH (carboxyl), —CONH₂ (carboxamide), —SO₃H (sulfa) and —CN (cyano). Functional groups and heteroatoms may also be directly adjacent, and so combinations of a plurality of adjacent atoms, for instance —O— (ether), —S— (thioether), —COO— (ester), —CONH— (secondary amide) or —CONR′— (tertiary amide) are also included, for example di(C₁-C₄-alkyl)amino, C₁-C₄-alkyloxycarbonyl or C₁-C₄-alkyloxy. The R′ radicals are the remaining part of the carbon-comprising radical.

Halogens include fluorine, chlorine, bromine and iodine.

The R radical is preferably
unbranched or branched, unsubstituted or mono- to poly-hydroxyl-, -halogen-, -phenyl-, -cyano-, —C₁-C₆-alkoxycarbonyl- and/or —SO₃H-substituted C₁-C₁₈-alkyl with a total of 1 to 20 carbon atoms, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, benzyl, 3-phenylpropyl, 2-hydroxyethyl, 2-cyanoethyl, 2-(methoxycarbonyl)ethyl, 2-(ethoxycarbonyl)ethyl, 2-(n-butoxycarbonyl)ethyl, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl, 6-hydroxyhexyl and sulfopropyl;
glycols, butylene glycols and oligomers thereof having 1 to 100 units and a hydrogen or a C₁-C₈-alkyl as the end group, for example R^{A}O—(CHR^{B}—CH₂—O)ₙ—CHR^{B}—CH₂— or R^{A}O—(CH₂CH₂CH₂CH₂O)ₙ—CH₂CH₂CH₂CH₂O— where R^{A} and R^{B} are preferably each hydrogen, methyl or ethyl, and n is preferably 0 to 3, especially 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl;
vinyl;
1-propen-1-yl, 1-propen-2-yl and 1-propen-3-yl; and
N,N-di-C₁-C₆-alkylamino, for example N,N-dimethylamino and N,N-diethylamino.

The R radical is more preferably unbranched and unsubstituted C₁-C₁₈-alkyl, for example methyl, ethyl, 1-propyl, 1-butyl, 1-pentyl, 1-hexyl, 1-heptyl, 1-octyl, 1-decyl, 1-dodecyl, 1-tetradecyl, 1-hexadecyl, 1-octadecyl, especially methyl, ethyl, 1-butyl and 1-octyl, and also CH₃O—(CH₂CH₂O)ₙ—CH₂CH₂— and CH₃CH₂O—(CH₂CH₂O)ₙ—CH₂CH₂— where n is 0 to 3.

The R¹ to R⁹ radicals are preferably each independently
hydrogen;
halogen;
a functional group;
C₁-C₁₈-alkyl optionally substituted by functional groups, aryl, alkyl, aryloxy, alkyloxy, halogen, heteroatoms and/or heterocycles, and/or interrupted by one or more oxygen and/or sulfur atoms and/or one or more substituted or unsubstituted imino groups;
C₂-C₁₈-alkenyl optionally substituted by functional groups, aryl, alkyl, aryloxy, alkyloxy, halogen, heteroatoms and/or heterocycles, and/or interrupted by one or more oxygen and/or sulfur atoms and/or one or more substituted or unsubstituted imino groups;
C₈-C₁₂-aryl optionally substituted by functional groups, aryl, alkyl, aryloxy, alkyloxy, halogen, heteroatoms and/or heterocycles;
C₅-C₁₂-cycloalkyl optionally substituted by functional groups, aryl, alkyl, aryloxy, alkyloxy, halogen, heteroatoms and/or heterocycles;
C₅-C₁₂-cycloalkenyl optionally substituted by functional groups, aryl, alkyl, aryloxy, alkyloxy, halogen, heteroatoms and/or heterocycles; or
a five- to six-membered heterocycle having oxygen, nitrogen and/or sulfur atoms, optionally substituted by functional groups, aryl, alkyl, aryloxy, alkyloxy, halogen, heteroatoms and/or heterocycles; or

two adjacent radicals together with the atoms to which they are bonded are
an unsaturated, saturated or aromatic ring optionally substituted by functional groups, aryl, alkyl, aryloxy, alkyloxy, halogen, heteroatoms and/or heterocycles, and optionally interrupted by one or more oxygen and/or sulfur atoms and/or one or more substituted or unsubstituted imino groups.

C₁-C₁₈-alkyl optionally substituted by functional groups, aryl, alkyl, aryloxy, alkyloxy, halogen, heteroatoms and/or heterocycles is preferably methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl, cyclopentylmethyl, 2-cyclopentylethyl, 3-cyclopentylpropyl, cyclohexylmethyl, 2-cyclohexylethyl, 3-cyclohexylpropyl, benzyl (phenylmethyl), diphenylmethyl (benzhydryl), triphenylmethyl, 1-phenylethyl, 2-phenylethyl, 3-phenylpropyl, α,α-dimethylbenzyl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di-(methoxycarbonyl)ethyl, methoxy, ethoxy, formyl, 1,3-dioxolan-2-yl, 1,3-dioxan-2-yl, 2-methyl-1,3-dioxolan-2-yl, 4-methyl-1,3-dioxolan-2-yl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl, 6-ethoxyhexyl, acetyl, CₙF_{2(n-a)+(1-b)}H_{2a+b} where n is 1 to 30, 0≦a≦n and b=0 or 1 (for example CF₃, C₂F₅, CH₂CH₂—C₍ₙ₋₂₎F₂₍ₙ₋₂₎₊₁, C₆F₁₃, C₈F₁₇, C₁₀F₂₁, C₁₂F₂₅), chloromethyl, 2-chloroethyl, trichloromethyl, 1,1-dimethyl-2-chloroethyl, methoxymethyl, 2-butoxyethyl, diethoxymethyl, diethoxyethyl, 2-isopropoxyethyl, 2-butoxypropyl, 2-octyloxyethyl, 2-methoxyisopropyl, 2-(methoxycarbonyl)ethyl, 2-(ethoxycarbonyl)ethyl, 2-(n-butoxycarbonyl)ethyl, butylthiomethyl, 2-dodecylthioethyl, 2-phenylthioethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-dioxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-dioxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

C₂-C₁₈-Alkenyl optionally substituted by functional groups, aryl, alkyl, aryloxy, alkyloxy, halogen, heteroatoms and/or heterocycles and/or interrupted by one or more oxygen and/or sulfur atoms and/or one or more substituted or unsubstituted imino groups is preferably vinyl, 2-propenyl, 3-butenyl, cis-2-butenyl, trans-2-butenyl or CₙF_{2(n-a)-(1-b)}H_{2a-b} where n≦30, 0≦a≦n and b=0 or 1.

C₅-C₁₂-Aryl optionally substituted by functional groups, aryl, alkyl, aryloxy, alkyloxy, halogen, heteroatoms and/or heterocycles is preferably phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2-nitrophenyl, 4-nitrophenyl, 2,4-dinitrophenyl, 2,6-dinitrophenyl, 4-dimethylaminophenyl, 4-acetylphenyl, methoxyethylphenyl, ethoxymethylphenyl, methylthiophenyl, isopropylthiophenyl or tert-butylthiophenyl or C₆F₍₅₋ₐ₎Hₐ where 0≦a≦5.

C₅-C₁₂-Cycloalkyl optionally substituted by functional groups, aryl, alkyl, aryloxy, alkyloxy, halogen, heteroatoms and/or heterocycles is preferably cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl, CₙF_{2(n-a)-(1-b)}H_{2a-b} where n≦30, 0≦a≦n and b=0 or 1, or a saturated or unsaturated bicyclic system such as norbornyl or norbornenyl.

C₅- to C₁₂-cycloalkenyl optionally substituted by functional groups, aryl, alkyl, aryloxy, alkyloxy, halogen, heteroatoms and/or heterocycles is preferably 3-cyclopentenyl, 2-cyclohexenyl, 3-cyclohexenyl, 2,5-cyclohexadienyl or CₙF_{2(n-a)-3(1-b)}H_{2a-3b} where n≦30, 0≦a≦n and b=0 or 1.

A five- or six-membered, oxygen-, nitrogen- and/or sulfur-comprising heterocycle optionally substituted by functional groups, aryl, alkyl, aryloxy, alkyloxy, halogen, heteroatoms and/or heterocycles is preferably furyl, thiophenyl, pyrryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl or difluoropyridyl.

When two adjacent radicals together form an unsaturated, saturated or aromatic ring optionally substituted by functional groups, aryl, alkyl, aryloxy, alkyloxy, halogen, heteroatoms and/or heterocycles and optionally interrupted by one or more oxygen and/or sulfur atoms and/or one or more substituted or unsubstituted imino groups, the ring is preferably 1,3-propylene, 1,4-butylene, 1,5-pentylene, 2-oxa-1,3-propylene, 1-oxa-1,3-propylene, 2-oxa-1,3-propylene, 1-oxa-1,3-propenylene, 3-oxa-1,5-pentylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

When the abovementioned radicals comprise oxygen and/or sulfur atoms and/or substituted or unsubstituted imino groups, the number of oxygen and/or sulfur atoms and/or imino groups is not restricted. In general, there will be no more than 5 in the radical, preferably no more than 4 and very particularly preferably no more than 3.

When the abovementioned radicals comprise heteroatoms, there is generally at least one carbon atom, preferably at least two carbon atoms, between any two heteroatoms.

The R¹ to R⁹ radicals are more preferably each independently
hydrogen;
unbranched or branched C₁-C₁₈-alkyl which is unsubstituted or substituted by one or more hydroxyl, halogen, phenyl, cyano, C₁-C₆-alkoxycarbonyl and/or SO₃H and has a total of from 1 to 20 carbon atoms, for example methyl, ethyl, 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, benzyl, 3-phenylpropyl, 2-hydroxyethyl, 2-cyanoethyl, 2-(methoxycarbonyl)ethyl, 2-(ethoxycarbonyl)ethyl, 2-(n-butoxycarbonyl)ethyl, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl, 6-hydroxyhexyl and sulfopropyl;
glycols, butylene glycols and oligomers thereof having from 1 to 100 units and a hydrogen or a C₁-C₈-alkyl as the end group, for example R^{A}O—(CHR^{B}—CH₂—O)ₙ—CHR^{B}—CH₂— or R^{A}O—(CH₂CH₂CH₂CH₂O)ₙ—CH₂CH₂CH₂CH₂O— where R^{A} and R^{B} are preferably each hydrogen, methyl or ethyl and n is preferably 0 to 3, especially 3-oxabutyl, 3-oxapentyl, 3,6-dioxaheptyl, 3,6-dioxaoctyl, 3,6,9-trioxadecyl, 3,6,9-trioxaundecyl, 3,6,9,12-tetraoxamidecyl and 3,6,9,12-tetraoxatetradecyl;
vinyl;
1-propen-1-yl, 1-propen-2-yl and 1-propen-3-yl; and
N,N-di-C₁-C₆-alkylamino, for example N,N-dimethylamino and N,N-diethylamino.

The R¹ to R⁹ radicals are most preferably each independently hydrogen or C₁-C₁₈-alkyl, for example methyl, ethyl, 1-butyl, 1-pentyl, 1-hexyl, 1-heptyl, 1-octyl, phenyl, 2-hydroxyethyl, 2-cyanoethyl, 2-(methoxycarbonyl)ethyl, 2-(ethoxycarbonyl)ethyl, 2-(n-butoxycarbonyl)ethyl, N,N-dimethylamino, N,N-diethylamino, chlorine or CH₃O—(CH₂CH₂O)ₙ—CH₂CH₂— and CH₃CH₂O—(CH₂CH₂O)ₙ—CH₂CH₂— where n is 0 to 3.

The pyridinium ions (IVa) used are most preferably those in which
one of the R¹ to R⁵ radicals is methyl, ethyl or chlorine and the remaining R¹ to R⁵ radicals are each hydrogen;
R³ is dimethylamino and the remaining R¹, R², R⁴ and R⁵ radicals are each hydrogen;
all R¹ to R⁵ radicals are hydrogen;
R² is carboxyl or carboxamide and the remaining R¹, R², R⁴ and R⁵ radicals are each hydrogen; or
R¹ and R² or R² and R³ together are 1,4-buta-1,3-dienylene and the remaining R¹, R², R⁴ and R⁵ radicals are each hydrogen;

and especially those in which
R¹ to R⁵ are each hydrogen; or
one of the R¹ to R⁵ radicals is methyl or ethyl and the remaining R¹ to R⁵ radicals are each hydrogen.

Very particularly preferred pyridinium ions (IVa) include 1-methylpyridinium, 1-ethylpyridinium, 1-(1-butyl)pyridinium, 1-(1-hexyl)pyridinium, 1-(1-octyl)pyridinium, 1-(1-hexyl)pyridinium, 1-(1-octyl)pyridinium, 1-(1-dodecyl)pyridinium, 1-(1-tetradecyl)-pyridinium, 1-(1-hexadecyl)pyridinium, 1,2-dimethylpyridinium, 1-ethyl-2-methyl-pyridinium, 1-(1-butyl)-2-methylpyridinium, 1-(1-hexyl)-2-methylpyridinium, 1-(1-octyl)-2-methylpyridinium, 1-(1-dodecyl)-2-methylpyridinium, 1-(1-tetradecyl)-2-methylpyridinium, 1-(1-hexadecyl)-2-methylpyridinium, 1-methyl-2-ethylpyridinium, 1,2-diethylpyridinium, 1-(1-butyl)-2-ethylpyridinium, 1-(1-hexyl)-2-ethylpyridinium, 1-(1-octyl)-2-ethylpyridinium, 1-(1-dodecyl)-2-ethylpyridinium, 1-(1-tetradecyl)-2-ethylpyridinium, 1-(1-hexadecyl)-2-ethylpyridinium, 1,2-dimethyl-5-ethylpyridinium, 1,5-diethyl-2-methylpyridinium, 1-(1-butyl)-2-methyl-3-ethylpyridinium, 1-(1-hexyl)-2-methyl-3-ethylpyridinium and 1-(1-octyl)-2-methyl-3-ethylpyridinium, 1-(1-dodecyl)-2-methyl-3-ethylpyridinium, 1-(1-tetradecyl)-2-methyl-3-ethylpyridinium and 1-(1-hexadecyl)-2-methyl-3-ethylpyridinium.

The pyridazinium ions (IVb) used are most preferably those in which
R¹ to R⁴ are each hydrogen; or
one of the R¹ to R⁴ radicals is methyl or ethyl and the remaining R¹ to R⁴ radicals are each hydrogen.

The pyrimidinium ions (IVc) used are most preferably those in which
R¹ is hydrogen, methyl or ethyl and R² to R⁴ are each independently hydrogen or methyl; or
R¹ is hydrogen, methyl or ethyl, R² and R⁴ are each methyl and R³ is hydrogen.

The pyrazinium ions (IVd) used are most preferably those in which
R¹ is hydrogen, methyl or ethyl and R² to R⁴ are each independently hydrogen or methyl;
R¹ is hydrogen, methyl or ethyl, R² and R⁴ are each methyl and R³ is hydrogen;
R¹ to R⁴ are each methyl; or
R¹ to R⁴ are each hydrogen.

The imidazolium ions (IVe) used are most preferably those in which
R¹ is hydrogen, methyl, ethyl, 1-propyl, 1-butyl, 1-pentyl, 1-hexyl, 1-octyl, 2-hydroxyethyl or 2-cyanoethyl and R² to R⁴ are each independently hydrogen, methyl or ethyl.

Very particularly preferred imidazolium ions (IVe) include 1-methylimidazolium, 1-ethylimidazolium, 1-(1-butyl)imidazolium, 1-(1-octyl)imidazolium, 1-(1-dodecyl)imidazolium, 1-(1-tetradecyl)imidazolium, 1-(1-hexadecyl)imidazolium, 1,3-dimethylimidazolium, 1-ethyl-3-methylimidazolium, 1-(1-butyl)-3-methylimidazolium, 1-(1-butyl)-3-ethylimidazolium, 1-(1-hexyl)-3-methylimidazolium, 1-(1-hexyl)-3-ethylimidazolium, 1-(1-hexyl)-3-butylimidazolium, 1-(1-octyl)-3-methylimidazolium, 1-(1-octyl)-3-ethylimidazolium, 1-(1-octyl)-3-butylimidazolium, 1-(1-dodecyl)-3-methylimidazolium, 1-(1-dodecyl)-3-ethylimidazolium, 1-(1-dodecyl)-3-butylimidazolium, 1-(1-dodecyl)-3-octylimidazolium, 1-(1-tetradecyl)-3-methylimidazolium, 1-(1-tetradecyl)-3-ethylimidazolium, 1-(1-tetradecyl)-3-butylimidazolium, 1-(1-tetradecyl)-3-octylimidazolium, 1-(1-hexadecyl)-3-methylimidazolium, 1-(1-hexadecyl)-3-ethylimidazolium, 1-(1-hexadecyl)-3-butylimidazolium, 1-(1-hexadecyl)-3-octylimidazolium, 1,2-dimethylimidazolium, 1,2,3-trimethylimidazolium, 1-ethyl-2,3-dimethylimidazolium, 1-(1-butyl)-2,3-dimethylimidazolium, 1-(1-hexyl)-2,3-dimethylimidazolium, 1-(1-octyl)-2,3-dimethylimidazolium, 1,4-dimethylimidazolium, 1,3,4-trimethylimidazolium, 1,4-dimethyl-3-ethylimidazolium, 3-butylimidazolium, 1,4-dimethyl-3-octylimidazolium, 1,4,5-trimethylimidazolium, 1,3,4,5-tetramethylimidazolium, 1,4,5-trimethyl-3-ethyl-imidazolium, 1,4,5-trimethyl-3-butylimidazolium, 1,4,5-trimethyl-3-octylimidazolium, and 1-(prop-1-en-3-yl)-3-methylimidazolium.

The pyrazolium ions (IVf), (IVg) and (IVg′) used are most preferably those in which
R¹ is hydrogen, methyl or ethyl and R² to R⁴ are each independently hydrogen or methyl.

The pyrazolium ions (IVh) used are most preferably those in which
R¹ to R⁴ are each independently hydrogen or methyl.

The 1-pyrazolinium ions (IVi) used are most preferably those in which
R¹ to R⁶ are each independently hydrogen or methyl.

The 2-pyrazolinium ions (IVj) and (IVj′) used are most preferably those in which
R¹ is hydrogen, methyl, ethyl or phenyl and R² to R⁶ are each independently hydrogen or methyl.

The 3-pyrazolinium ions (IVk) and (IVk′) used are most preferably those in which
R¹ and R² are each independently hydrogen, methyl, ethyl or phenyl and R³ to R⁶ are each independently hydrogen or methyl.

The imidazolinium ions (IVl) used are most preferably those in which
R¹ and R² are each independently hydrogen, methyl, ethyl, 1-butyl or phenyl, R³ and R⁴ are each independently hydrogen, methyl or ethyl and R⁵ and R⁶ are each independently hydrogen or methyl.

The imidazolinium ions (IVm) and (IVm′) used are most preferably those in which
R¹ and R² are each independently hydrogen, methyl or ethyl and R³ to R⁶ are each independently hydrogen or methyl.

The imidazolinium ions (IVn) and (IVn′) used are most preferably those in which
R¹ to R³ are each independently hydrogen, methyl or ethyl and R⁴ to R⁶ are each independently hydrogen or methyl.

The thiazolium ions (IVo) and (IVo′) and oxazolium ions (IVp) used are most preferably those in which
R¹ is hydrogen, methyl, ethyl or phenyl and R² and R³ are each independently hydrogen or methyl.

The 1,2,4-triazolium ions (IVq), (IVq′) and (IVq″) used are most preferably those in which
R¹ and R² are each independently hydrogen, methyl, ethyl or phenyl and R³ is hydrogen, methyl or phenyl.

The 1,2,3-triazolium ions (IVr), (IVr′) and (IVr″) used are most preferably those in which
R¹ is hydrogen, methyl or ethyl and R² and R³ are each independently hydrogen or methyl or R² and R³ together are 1,4-buta-1,3-dienylene.

The pyrrolidinium ions (IVs) used are most preferably those in which
R¹ is hydrogen, methyl, ethyl or phenyl and R² to R⁹ are each independently hydrogen or methyl.

The imidazolidinium ions (IVt) used are most preferably those in which
R¹ and R⁴ are each independently hydrogen, methyl, ethyl or phenyl and R² and R³ and also R⁵ to R⁸ are each independently hydrogen or methyl.

The ammonium ions (IVu) used are most preferably those in which
R¹ to R³ are each independently C₁-C₁₈-alkyl; or
R¹ and R² together are 1,5-pentylene or 3-oxa-1,5-pentylene and R³ is C₁-C₁₈-alkyl, 2-hydroxyethyl or 2-cyanoethyl.

Very particularly preferred ammonium ions (IVu) include trimethylammonium, triethylammonium, dimethylethylammonium, diethylmethylammonium, tetramethylammonium.

Examples of the tertiary amines from which the quaternary ammonium ions of the general formula (IVu) derive by quaternization by the R radicals mentioned are trimethylamine, triethylamine, tri-n-propylamine, tri-n-butylamine, diethylmethylamine, dimethylethylamine, triisopropylamine, isopropyldiethylamine, diisopropylethylamine, diethyl-n-butylamine, diethyl-tert-butylamine, diethyl-n-pentylamine, diethylhexylamine, diethyloctylamine, diethyl-(2-ethylhexyl)amine, di-n-propylbutylamine, di-n-propyl-n-pentylamine, di-n-propylhexylamine, di-n-propyloctylamine, di-n-propyl-(2-ethyl-hexyl)amine, diisopropylethylamine, diisopropyl-n-propylamine, diisopropylbutylamine, diisopropylpentylamine, diisopropylhexylamine, diisopropyloctylamine, diisopropyl(2-ethylhexyl)amine, di-n-butylethylamine, di-n-butyl-n-propylamine, di-n-butyl-n-pentylamine, di-n-butylhexylamine, di-n-butyloctylamine, di-n-butyl(2-ethylhexyl)amine, N-n-butylpyrrolidine, N-sec-butylpyrrolidine, N-tert-butylpyrrolidine, N-n-pentylpyrrolidine, N,N-dimethylcyclohexylamine, N,N-diethylcyclohexylamine, N,N-di-n-butylcyclohexylamine, N-n-propylpiperidine, N-isopropylpiperidine, N-n-butylpiperidine, N-sec-butylpiperidine, N-tert-butylpiperidine, N-n-pentylpiperidine, N-n-butylmorpholine, N-sec-butylmorpholine, N-tert-butylmorpholine, N-n-pentylmorpholine, N-benzyl-N-ethylaniline, N-benzyl-N-n-propylaniline, N-benzyl-N-isopropylaniline, N-benzyl-N-n-butylaniline, N,N-dimethyl-p-toluidine, N,N-diethyl-p-toluidine, N,N-di-n-butyl-p-toluidine, diethylbenzylamine, di-n-propylbenzylamine, di-n-butylbenzylamine, diethylphenylamine, di-n-propylphenylamine and di-n-butylphenylamine.

Preferred quaternary ammonium salts of the general formula (IVu) are those which can be derived from the following tertiary amines by quaternization with the R radicals mentioned: dimethylamine, trimethylamine, diethylamine, triethylamine, dimethylethylamine, diethyl-tert-butylamine, diisopropylethylamine, tripropylamine, tributylamine.

Particularly preferred tertiary amines are trimethylamine and triethylamine.

The guanidinium ions (IVv) used are most preferably those in which
R, to R⁵ are each methyl.

A very particularly preferred guanidinium ion (IVv) is N,N,N′,N′,N″,N″-hexamethylguanidinium.

The cholinium ions (IVw) used are most preferably those in which
R¹ and R² are each independently methyl, ethyl, 1-butyl or 1-octyl and R³ is hydrogen, methyl, ethyl, acetyl, —SO₂OH or —PO(OH)₂;
R¹ is methyl, ethyl, 1-butyl or 1-octyl, R² is a —CH₂—CH₂—OR⁴ group and R³ and R⁴ are each independently hydrogen, methyl, ethyl, acetyl, —SO₂OH or —PO(OH)₂; or
R¹ is a —CH₂—CH₂—OR⁴ group, R² is a —CH₂—CH₂—OR⁵ group and R³ to R⁵ are each independently hydrogen, methyl, ethyl, acetyl, —SO₂OH or —PO(OH)₂.

Particularly preferred cholinium ions (IVw) are those in which R³ is selected from among hydrogen, methyl, ethyl, acetyl, 5-methoxy-3-oxapentyl, 8-methoxy-3,6-dioxa-octyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

The phosphonium ions (IVx) used are most preferably those in which
R¹ to R³ are each independently C₁-C₁₈-alkyl, especially butyl, isobutyl, 1-hexyl or 1-octyl.

Among the abovementioned heterocyclic cations, preference is given to the pyridinium ions, pyrazolinium ions, pyrazolium ions and the imidazolinium ions and the imidazolium ions. Preference is also given to ammonium ions.

Especially preferred are 1-methylpyridinium, 1-ethylpyridinium, 1-(1-butyl)pyridinium, 1-(1-hexyl)pyridinium, 1-(1-octyl)pyridinium, 1-(1-hexyl)pyridinium, 1-(1-octyl)pyridinium, 1-(1-dodecyl)pyridinium, 1-(1-tetradecyl)pyridinium, 1-(1-hexadecyl)pyridinium, 1,2-dimethylpyridinium, 1-ethyl-2-methylpyridinium, 1-(1-butyl)-2-methylpyridinium, 1-(1-hexyl)-2-methylpyridinium, 1-(1-octyl)-2-methylpyridinium, 1-(1-dodecyl)-2-methylpyridinium, 1-(1-tetradecyl)-2-methylpyridinium, 1-(1-hexadecyl)-2-methylpyridinium, 1-methyl-2-ethylpyridinium, 1,2-diethylpyridinium, 1-(1-butyl)-2-ethylpyridinium, 1-(1-hexyl)-2-ethylpyridinium, 1-(1-octyl)-2-ethylpyridinium, 1-(1-dodecyl)-2-ethylpyridinium, 1-(1-tetradecyl)-2-ethylpyridinium, 1-(1-hexadecyl)-2-ethylpyridinium, 1,2-dimethyl-5-ethylpyridinium, 1,5-diethyl-2-methylpyridinium, 1-(1-butyl)-2-methyl-3-ethylpyridinium, 1-(1-hexyl)-2-methyl-3-ethylpyridinium, 1-(1-octyl)-2-methyl-3-ethylpyridinium, 1-(1-dodecyl)-2-methyl-3-ethylpyridinium, 1-(1-tetradecyl)-2-methyl-3-ethylpyridinium, 1-(1-hexadecyl)-2-methyl-3-ethylpyridinium, 1-methylimidazolium, 1-ethylimidazolium, 1-(1-butyl)imidazolium, 1-(1-octyl)imidazolium, 1-(1-dodecyl)imidazolium, 1-(1-tetradecyl)imidazolium, 1-(1-hexadecyl)imidazolium, 1,3-dimethylimidazolium, 1-ethyl-3-methylimidazolium, 1-(1-butyl)-3-methylimidazolium, 1-(1-hexyl)-3-methylimidazolium, 1-(1-octyl)-3-methylimidazolium, 1-(1-dodecyl)-3-methylimidazolium, 1-(1-tetradecyl)-3-methylimidazolium, 1-(1-hexadecyl)-3-methylimidazolium, 1,2-dimethylimidazolium, 1,2,3-trimethylimidazolium, 1-ethyl-2,3-dimethylimidazolium, 1-(1-butyl)-2,3-dimethylimidazolium, 1-(1-hexyl)-2,3-dimethylimidazolium and 1-(1-octyl)-2,3-dimethylimidazolium, 1,4-dimethylimidazolium, 1,3,4-trimethylimidazolium, 1,4-dimethyl-3-ethylimidazolium, 3-butylimidazolium, 1,4-dimethyl-3-octylimidazolium, 1,4,5-trimethylimidazolium, 1,3,4,5-tetramethylimidazolium, 1,4,5-trimethyl-3-ethylimidazolium, 1,4,5-trimethyl-3-butylimidazolium, 1,4,5-trimethyl-3-octylimidazolium and 1-(prop-1-en-3-yl)-3-methylimidazolium.

The metal cations [M¹]⁺, [M²]⁺, [M³]⁺, [M⁴]²⁺ and [M⁵]³⁺ mentioned in the formulae (IIIa) to (IIIj) are generally metal cations of groups 1., 2., 6., 7., 8., 9., 10., 11., 12. and 13. of the periodic table. Suitable metal cations are, for example, Li⁺, Na⁺, K⁺, Cs⁺, Mg²⁺, Ca²⁺, Ba²⁺, Cr³⁺, Fe²⁺, Fe³⁺, Co²⁺, Ni²⁺, Cu²⁺, Ag⁺, Zn²⁺ and Al³⁺.

The organic cation is more preferably an ammonium ion, optionally C₁₋₄-alkyl-substituted pyridinium ion or optionally C₁₋₄-alkyl-substituted imidazolium ion.

The organic cation is most preferably a trimethylammonium ion, triethylammonium ion, unsubstituted pyridinium ion or 1-ethyl-3-methylimidazolium ion.

The anion of the ionic liquids used in accordance with the invention is selected from superacidic aluminum trichloride-Lewis base adducts. Aluminum trichloride (AlCl₃) is a Lewis acid.

In the context of the present invention, the expression “superacidic aluminum trichloride-Lewis base adducts” refers to those aluminum trichloride-Lewis base adducts which, in protonated form, have a pKₐ which is less than that of a strong acid or less than or equal to the pKₐ of an extremely strong acid. The superacidic aluminum trichloride-Lewis base adducts used in accordance with the invention preferably have, in protonated form, a pKₐ of <−7, i.e. a smaller pKₐ than HCl.

In the context of the present invention, the expression “aluminum trichloride-Lewis base adduct” refers to complex anions which are formed by the addition of an anion, especially of a chloride or bromide, onto the Lewis acid aluminum trichloride. The addition products may also form adducts with one or two further (identical or different) Lewis acid molecules.

Typically, suitable Lewis acid-Lewis base adducts are selected from compounds of the formula [MetₐZ_{b}]⁻, in which the value of b corresponds to the product of oxidation number of the metal or semimetal Met and the index a, plus 1, i.e. b=a·Ox+1, where Ox is the oxidation number of the metal or semimetal. Typically, a has a value in the range from 1 to 3. Preferably, a in the Lewis acid-Lewis base adducts is 2 or 3.

When a is 2 or 3, the metals or semimetals Met present in the Lewis acid-Lewis base adduct may be the same or different. Lewis acid-Lewis base adducts with different metals form, for example, when a Lewis acid-Lewis base adduct first forms from a Lewis acid and a halide ion, and is then reacted with a further Lewis acid other than the first Lewis acid to form an adduct. Preferably, however, all Met present in the Lewis acid-Lewis base adduct [MetₐZ_{b}]⁻ are the same, and are Al.

In the Lewis acid-Lewis base adduct of the formula [MetₐZ_{b}]⁻, Z may be the same or different. Lewis acid-Lewis base adducts with mixed Z are obtained, for example, when, as described above, the Lewis acid-Lewis base adduct forms from two different Lewis acids. Alternatively, it is obtained when Lewis acids with mixed halogen atoms are used, or when the halide ion which functions as a Lewis base is different than the halogen atom of the Lewis acid. All Z present in the Lewis acid-Lewis base adduct of the formula [MetₐZ_{b}]⁻ are especially the same; Z is especially chlorine or bromine.

Examples of suitable Lewis bases are Cl⁻, Br⁻, AlCl₄⁻, AlBrCl₃⁻, Al₂Cl₇⁻, Al₂BrCl₆⁻., Al₃Cl₁₀⁻, Al₃BrCl₉⁻, BCl₄⁻, BBr₄⁻, TiCl₅⁻, VCl₆⁻, FeCl₄⁻, FeBr₄⁻, Fe₂Cl₇⁻, Fe₃Cl₁₀⁻, ZnCl₃⁻, ZnBr₃⁻, CuCl₂⁻, CuBr₂⁻, CuCl₃⁻, CuBr₃⁻, NbCl₆⁻, SnCl₃⁻, SnBr₃⁻, SnCl₅⁻, SnBr₅⁻ and (CF₃SO₂)₂N⁻.

Preferred Lewis bases are AlCl₄⁻, Al₂Cl₇⁻, BCl₄⁻, BBr₄⁻, TiCl₅⁻, FeCl₄⁻, FeBr₄⁻, Fe₂Cl₇⁻ and Fe₃Cl₁₀⁻.

Accordingly, the anion of the ionic liquid is, for example, AlCl₄⁻, AlBrCl₃⁻, Al₂Cl₇⁻, Al₂BrCl₆⁻, Al₃Cl₁₀⁻, Al₃BrCl₉⁻ or (CF₃SO₂)₂NAlCl₃⁻.

Preferred anions Y⁻ are selected from AlBrCl₃⁻, Al₂Cl₇⁻, Al₂BrCl₆⁻, Al₃Cl₁₀⁻, Al₃BrCl₉⁻.

The anions Y⁻ are more preferably selected from Al₂Cl₇⁻, Al₃Cl₁₀⁻, and are especially Al₂Cl₇⁻.

Such an ionic liquid is prepared especially by adding the appropriate amount of aluminum chloride to the ionic liquid or to an ammonium chloride.

In the superacidic ionic liquid comprising an organic cation and an inorganic anion, where the anion is a superacidic aluminum trichloride-Lewis base adduct, the molar ratio of aluminum trichloride to Lewis base is preferably >1.0, particularly ≧1.5, more particularly ≧2.0. The molar ratio of aluminum trichloride to Lewis base is preferably ≦3.0, particularly ≦2.5, most preferably =2.0.

For the superacidic ionic liquid used in accordance with the invention, the Hammett function H₀ is preferably in the range from −16 to −20, particularly in the range from −17 to −19.

### EXAMPLES

### General Experimental Procedure

In each case, a 250 ml Miniplant stirred vessel with disc stirrer, internal thermometer, jacketed coil condenser, 200 ml feed vessel with Teflon tap (10 mm bore), thermometer and pressure equalizer to the jacketed coil condenser, stirrer driver with speed display, sampling attachment with tap and septum, and inertization with argon dried over sodium hydroxide, was used.

The ionic liquid (IL) (150 ml) was initially charged in the stirred vessel under argon, and the methylcyclopentane-containing organic mixture to be isomerized (30 ml) was introduced into the feed vessel. After heating all reactants to 60° C., the entire contents of the feed vessel were transferred by opening the Teflon tap within 1-2 seconds into the stirred vessel containing the IL while stirring. At defined time intervals, 5 ml of sample were taken each time by means of a 30 cm cannula and syringe through the sampling stub with septum. After approx. 2 minutes, the lighter organic phase which had separated out was introduced from the syringe into approx. 5 ml of 10% by weight aqueous sodium-EDTA solution and shaken. The phases were then separated. The organic phase was diluted with 2 ml of methylene chloride and, after drying with anhydrous sodium sulfate, analyzed by means of GC.

The examples which follow relate to the isomerization of methylcyclopentane to cyclohexane.

### Abbreviations Used:

RE: reaction equilibrium
MCP: methylcyclopentane
CH: cyclohexane
TMA: trimethylammonium
EMIM: 1-ethyl-3-methylimidazolium

### Example 1

### Comparative

### IL: TMA-Al₂Cl₇

Organic phase: pure MCP
Time until attainment of RE (80% MCP conversion): 180 min

### Example 2

### Inventive

### IL: TMA-Al₂Cl₇

Organic phase: pure MCP with 0.1% by weight of 2-methyl-1-pentene
Time until attainment of RE (80% MCP conversion): 70 min

### Example 3

### Inventive

### IL: TMA-Al₂Cl₇

Organic phase: pure MCP with 0.3% by weight of 2-methyl-1-pentene
Time until attainment of RE (80% MCP conversion): 40 min

### Example 4

### Comparative

### IL: EMIM-Al₂Cl₇

Organic phase: pure MCP
RE not attained after 180 min, MCP conversion after 180 min: 47%

### Example 5

### Inventive

### IL: EMIM-Al₂Cl₇

Organic phase: pure MCP with 0.3% by weight of 2-methyl-1-pentene
Time until attainment of RE (80% MCP conversion): 180 min

### Example 6

### Inventive

### IL: EMIM-Al₂Cl₇

Organic phase: pure MCP with 2% by weight of 2-methyl-1-pentene
Time until attainment of RE (80% MCP conversion): 70 min

### Example 7

### Comparative

### IL: TMA-Al₂Cl₇

Organic phase: 39% by weight of MCP, 12% by weight of CH, 49% by weight of n-hexane
Time until attainment of RE (77% MCP conversion): 300 min

### Example 8

### Inventive

### IL: TMA-Al₂Cl₇

Organic phase: 39% by weight of MCP, 12% by weight of CH, 49% by weight of n-hexane, 0.3% by weight of 2-methyl-1-pentene
Time until attainment of RE (77% MCP conversion): 70 min

### Example 9

### Inventive

### IL: TMA-Al₂Cl₇

Organic phase: 39% by weight of MCP, 12% by weight of CH, 47% by weight of n-hexane, 2% by weight of 2-methyl-1-pentene
Time until attainment of RE (77% MCP conversion): 5 min

### Example 10

### Comparative

### IL: EMIM-Al₂Cl₇

Organic phase: 39% by weight of MCP, 12% by weight of CH, 49% by weight of n-hexane
RE (77% MCP conversion) not attained after 180 min, MCP conversion after 180 min: 59%

### Example 11

### Inventive

### IL: EMIM-Al₂Cl₇

Organic phase: 39% by weight of MCP, 12% by weight of CH, 49% by weight of n-hexane, 0.1% by weight of 2-methyl-1-pentene
RE (77% MCP conversion) not attained after 180 min, MCP conversion after 180 min: 74%

### Example 12

### Inventive

### IL: EMIM-Al₂Cl₇

Organic phase: 39% by weight of MCP, 12% by weight of CH, 47% by weight of n-hexane, 2% by weight of 2-methyl-1-pentene
Time until attainment of RE (77% MCP conversion): 120 min

1.-19. (canceled)

20. A process for isomerizing a saturated hydrocarbon, which comprises performing the isomerization in the presence of a superacidic ionic liquid comprising an organic cation and an inorganic anion, where the anion is a superacidic aluminum trichloride-Lewis base adduct, and of an olefin.

21. The process according to claim 20, wherein the olefin is a linear or branched and/or cyclic C₂₋₁₄-olefin.

22. The process according to claim 20, wherein the olefin is a linear or branched and/or cyclic C₂₋₁₀-olefin.

23. The process according to claim 20, wherein the olefin is a monoolefin.

24. The process according to claim 20, wherein the olefin is ethene, 2-methyl-1-butene, 2-methyl-2-butene or 1-methylcyclopentene.

25. The process according to claim 20, wherein the isomerization is performed in the presence of 0.01 to 5% by weight of the olefin, based on the saturated hydrocarbon used.

26. The process according to claim 20, wherein the isomerization is performed in the presence of 0.1 to 3% by weight of the olefin, based on the saturated hydrocarbon used.

27. The process according claim 20, wherein the hydrocarbon to be isomerized is a linear or branched and/or cyclic C₄₋₁₈ hydrocarbon.

28. The process according to claim 20, wherein the hydrocarbon to be isomerized is a linear or branched and/or cyclic C₅₋₈ hydrocarbon.

29. The process according to claim 20, wherein a tertiary carbon atom of the hydrocarbon is converted to a secondary carbon atom in the course of isomerization.

30. The process according to claim 20, for isomerizing methylcyclopentane to cyclohexane.

31. The process according to claim 20, wherein the Hammett function H₀ for the superacidic ionic liquid comprising aluminum chloride is in the range from −16 to −20.

32. The process according to claim 20, wherein the organic cation is an ammonium ion, optionally C₁₋₄-alkyl-substituted pyridinium ion or optionally C₁₋₄-alkyl-substituted imidazolium ion.

33. The process according to claim 20, wherein the organic cation is a trimethylammonium ion, triethylammonium ion, unsubstituted pyridinium ion or 1-ethyl-3-methylimidazolium ion.

34. The process according to claim 20, wherein the inorganic anion is Al₂Cl₇⁻ or Al₂Cl₆Br⁻.

35. The process according to claim 20, wherein the isomerization is performed at a temperature in the range from −20 to 150° C.

36. The process according to claim 20, wherein the isomerization is performed at an absolute pressure in the range from 1 to 10 bar.

37. The process according to claim 20, wherein the hydrocarbon to be isomerized is used in a concentration in the range from 1 to 90% by weight, based on the ionic liquid.

38. The process according to claim 20, wherein the molar ratio of aluminum trichloride to Lewis base is >1.0 to ≦3.0.
